(19) United States
(12) Patent Application Publication
(10) Pub. No.: US 2022/0246284 A1
(43) Pub. Date: Aug. 4, 2022

(54) OUT-OF-HOSPITAL, IN FACILITY NON-EMERGENCY BLOOD AND BLOOD COMPONENT TRANSFUSION SERVICE

(71) Applicant: Sheryl Neelankavil Jose, Austin, TX (US)

(72) Inventor: Sheryl Neelankavil Jose, Austin, TX (US)

(21) Appl. No.: 17/163,501

(22) Filed: Jan. 31, 2021

(51) Int. Cl. G16H 40/20; A61M 1/02; G06Q 10/06; G16H 10/60; G16H 20/40; G16H 40/67

(52) U.S. Cl. CPC G16H 40/20; A61M 1/0286; G06Q 10/06315; A61M 2039/0205; G16H 20/40; G16H 40/67; A61M 2205/6009; G16H 10/60

(57) ABSTRACT

A method for providing remote blood-related healthcare, and more specifically, a method for providing blood transfusion treatment in a rehabilitation nursing home or skilled care facility is disclosed. The method receives call orders to provide services, dispatches to a skilled nursing facility to meet a patient, picks up blood products from a blood bank, travels to the skilled nursing facility, preforms pre-transfusion checks, transfuses blood products. and entering post transfusion document notes.

### TECHNICAL FIELD

This application relates in general to a method for providing remote blood-related healthcare, and more specifically, to a method for providing blood transfusion treatment in a rehabilitation nursing home or skilled care facility.

### BACKGROUND

Blood and blood component transfusion service currently can only be provided in an acute care hospital where the nursing home resident/chronically ill patient at home must be transferred to the hospital to perform the transfusion procedure. In today's healthcare setting, it is safe and cost effective for the nursing facility residents to receive blood and blood component transfusion at their bedside by a specially trained nurse rather than being shifted to an acute care hospital. Providing transportation to and monitoring and treatment in a hospital setting adds time, cost, complexity, and risk to the treatment of patients without improving the care received or outcome.

Therefore, a need exists for a transfusion service that is brought to a patient's bedside which includes a protocol developed with the local blood banks to provide the blood and blood components required for transfusion by a specially trained nurse.

### SUMMARY

In accordance with the present invention, the above and other problems are solved by providing a method for blood transfusion treatment in a rehabilitation nursing home or skilled care facility according to the principles and example embodiments disclosed herein.

In one embodiment, the present invention is a method for providing blood transfusion treatment in a rehabilitation nursing home or skilled care facility. The method receives call orders to provide services, dispatches to a skilled nursing facility to meet a patient, picks up blood products from a blood bank, travels to the skilled nursing facility, preforms pre-transfusion checks, transfuses blood products. and entering post transfusion document notes.

In another aspect of the present disclosure, the method further checks in with skilled nursing facility, confirms needed services for the patient, reviews the orders maintained at the skilled nursing facility, greets the patent and explains the transfusion procedure, determining whether any questions regarding required treatment, requests additional information regarding questions from skilled nursing facility administrator, and checks if patent possesses a blood band identification bracelet, and when missing, terminates the transfusion procedure.

In another aspect of the present disclosure, the preforming pre-transfusion checks verifies a patient ID, date-of-birth, and blood transfusion number, ensures presence of venous access device, obtains and records vital signs of the patient, checks patent condition, and when it is changing, terminates the transfusion procedure, determine whether patient has previously received a blood transfusion, ensures an available telephone is working, verifies patient consent form signed, and signs verification documentation.

In another aspect of the present disclosure, the preforming pre-transfusion checks further determines whether red blood cells are being transfused, and when red blood cells are being used, removes blood product from transporting contain and ice and wait twenty minutes for the blood product to adjust to room temperature, takes a photo of patient's type and cross bracelet and blood product container for documentation, compares patent type and cross bracelet and blood product container, and, terminating the transfusion procedure when they are in conflict, and when all pre-checks are completed satisfactorily, perform the transfusion.

In another aspect of the present disclosure, performing the transfusion includes documenting all notes and forms, administering any pre-transfusion medication, recording baseline patient vital signs, inserting needle to IV line, connecting blood product to the IV line, preparing saline solution and connecting to blood administration kit, starting transfusion and record date and start time, monitor patient vital signs until transfusion complete, and if patient experiences an adverse reaction before the transfusion is complete, terminating the transfusion and initiating adverse reaction protocol.

In another aspect of the present disclosure, entering post transfusion document notes includes flushing all IV lines with saline, obtaining and recording vital signs, discontinuing use of venous device, disposing all devices used in transfusion in sharps container, when an adverse reaction has been observed, contacting responsible nurse and ordering doctor, and complete all adverse reaction protocols, and completing notes and uploading notes and documents to remote server.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other methods for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only, and is not intended as a definition of the limits of the present invention.

### BRIEF DESCRIPTION OF THE DRAWINGS

Referring now to the drawings in which like reference numbers represent corresponding parts throughout:

FIG. 1 illustrates an example embodiment for a method that provides blood transfusion treatment in a rehabilitation nursing home or skilled care facility according to the present invention.

FIG. 2 illustrates a flowchart of an example embodiment of a method for providing blood transfusion treatment in a rehabilitation nursing home or skilled care facility according to the present invention.

FIG. 3 illustrates a flowchart for an example embodiment of an arrival and check in portion of a method for providing blood transfusion treatment in a rehabilitation nursing home or skilled care facility according to the present invention.

FIGS. 4a-b illustrate flowcharts for an example embodiment of pre- and post-transfusion checks before the blood product removal portion of a method for providing blood transfusion treatment in a rehabilitation nursing home or skilled care facility according to the present invention.

FIG. 5 illustrates a flowchart for an example embodiment of the blood and blood product transfusion portion of a method for providing blood transfusion treatment in a rehabilitation nursing home or skilled care facility according to the present invention.

FIG. 6 illustrates a flowchart for an example embodiment of the post-transfusion documentation and notes-generation and upload portion of a method for providing blood transfusion treatment in a rehabilitation nursing home or skilled care facility according to the present invention.

### DETAILED DESCRIPTION

This application relates in general a method for providing remote healthcare, and more specifically, to a method providing blood transfusion treatment in a rehabilitation nursing home or skilled care facility according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. Thus, for example, reference to “a needle” includes reference to one or more of such needles and “etching” includes one or more of such steps. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

As used herein, the term “about” means that dimensions, sizes, formulations, parameters, shapes, and other quantities and characteristics are not and need not be exact, but may be approximated and/or larger or smaller, as desired, reflecting tolerances, conversion factors, rounding off, measurement error and the like, and other factors known to those of skill. Further, unless otherwise stated, the term “about” shall expressly include “exactly,” consistent with the discussion above regarding ranges and numerical data.

The terms “customer,” “nurse,” and “user” refer to an entity, e.g. a human, using the blood transfusion process associated with the invention. The term user herein refers to one or more users.

The term “invention” or “present invention” refers to the invention being applied for via the patent application with the title “OUT-OF-HOSPITAL, IN FACILITY NON-EMERGENCY BLOOD AND BLOOD COMPONENT TRANSFUSION SERVICE.” The invention may be used interchangeably with “blood transfusion service.”

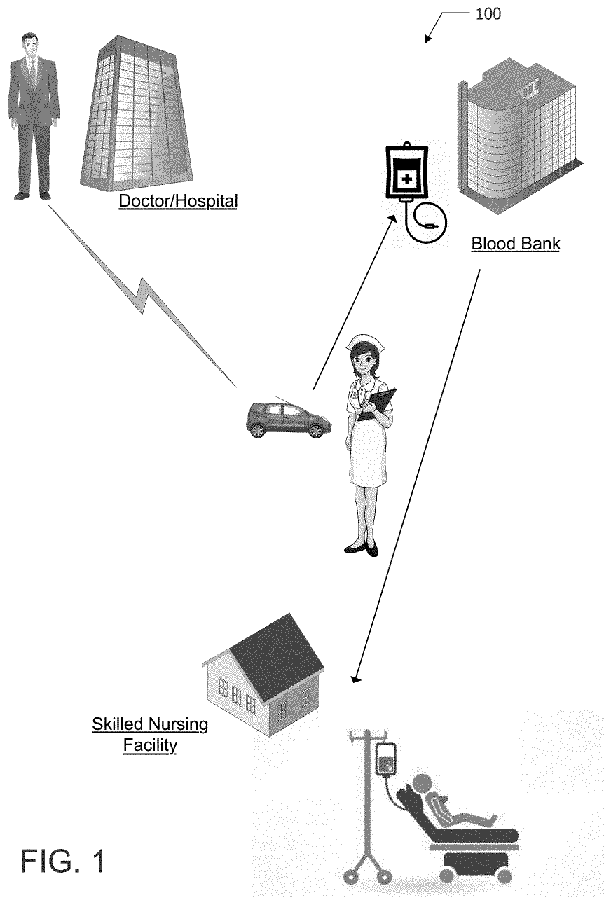

In general, the present disclosure relates a method for providing remote blood product healthcare treatment. To better understand the present invention, FIG. 1 illustrates an example embodiment for a method that provides blood transfusion treatment in a rehabilitation nursing home or skilled care facility according to the present invention. In this process, a skilled care nurse 101 receives a dispatch call from a doctor 102 or doctor's office 102a to perform a blood or blood product transfusion for a patient 110 at a rehabilitation or skilled care facility 111. The nurse 101 stops at a blood bank facility 104a to obtain the blood products 104 for the patient 110 before driving to the nursing home or skilled care facility 111. Once at the facility 111, the nurse 101 follows the process according to the present invention to administer the blood products 104 and provide remote, bed-side healthcare.

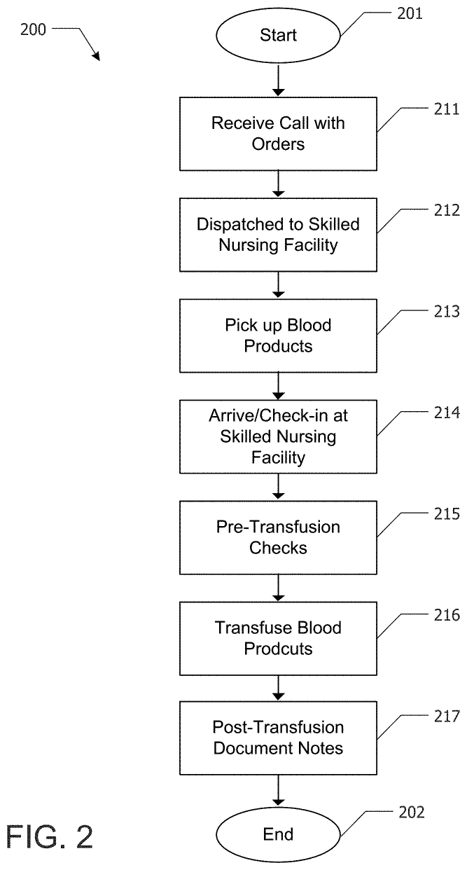

FIG. 2 illustrates a flowchart of an example embodiment of a method for providing blood transfusion treatment in a rehabilitation nursing home or skilled care facility according to the present invention. The process begins 201 when the nurse 101 receives a call for a physician-ordered transfusion and confirms with a nurse coordinator that the nurse 101 is taking the call in step 211. As part of confirming the call with the nurse coordinator, the nurse must update the mobile application with an updated status of one of the following: Scheduled, Dispatched, On the Way, On-Site, Delayed, Paused, Resumed (if necessary), Partially Completed, and Complete.

Before the transfusion can occur, the skilled nurse 101 performs a set of pre-transfusion checks, including: complete a pre-transfusion form, obtain an executed consent form, confirm a drawn patient's blood for type and cross sample, apply a blood transfusion identification bracelet to patient's wrist, add a copy of the completed pre-transfusion form and the executed consent form to the patient's chart, and write the blood transfusion identification number on the physician's orders. The skilled nurse 101 drops off the type and cross sample to a local blood bank, obtains a receipt from the blood bank for type and cross, and obtains an estimated time for blood pick up. All of the above information is uploaded to the patient's electronic medical record (EMR).

In step 212, the specially-trained nurse 101 has gone to meet with a patient. On the way to the patient, the nurse stops at the local blood bank in step 213 and picks up the blood or blood products. The nurse verifies the blood using the blood transfusion identification number and takes the blood in the blood bank's container that has been packed and prepared for the transfusion.

In step 214, the specially-trained nurse 101 arrives at the skilled care facility 111 and checks in with the staff and patient. The nurse 101 performs the pre-transfusion checks in step 215. The blood or blood product is transfused in step 216 and the nurse 101 completes and documents the procedure for upload to an EMR records system in step 216. Each of these steps is described in detail below.

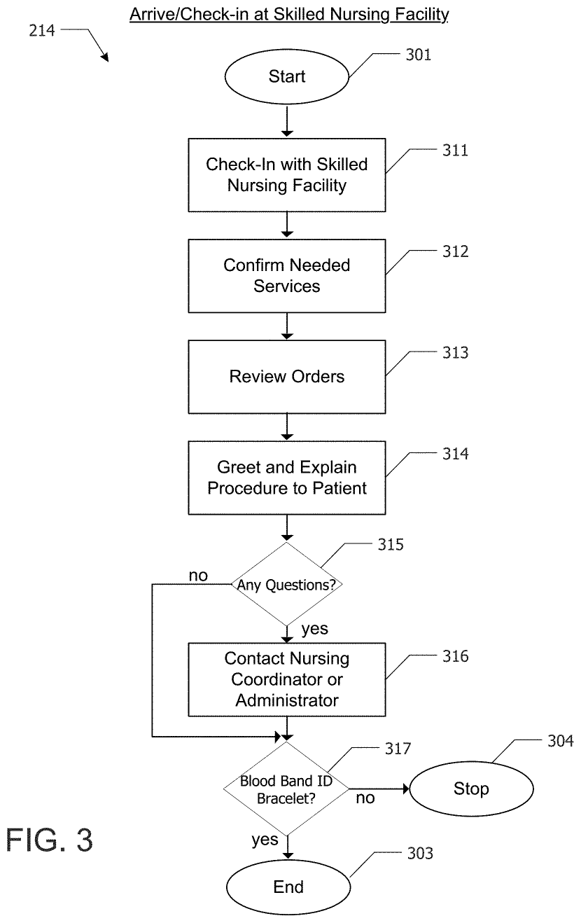

FIG. 3 illustrates a flowchart for an example embodiment of an arrival and check in portion step 214 of a method for providing blood transfusion treatment in a rehabilitation nursing home or skilled care facility according to the present invention. When the specially-trained nurse 101 arrives, the nurse 101 presents a specific badge identification that is displayed/worn during the time of service.

The specially-trained nurse 101 checks in with the facility in step 311 and confirms the type of service required in step 312. The nurse 101 reviews the physician's order in step 313. The nurse 101 then greets the patient in step 314 to explain the procedure.

Test step 315 determines whether or not the specially-trained nurse 101 has a question regarding the transfusion orders after all of the above initial checks are completed. If test step 315 determines that a question(s) exists, the nurse 101 contacts the nurse coordinator or administrator in step 316 prior to starting the procedure. The nurse 101 may also contact the facility or physician to review information if necessary.

Test step 317 determines if the blood band identification bracelet is present; if the identification band is absent, the specially-trained nurse 101 stops 304 and does NOT perform a transfusion, otherwise the procedure proceeds to the next step in the process, step 215.

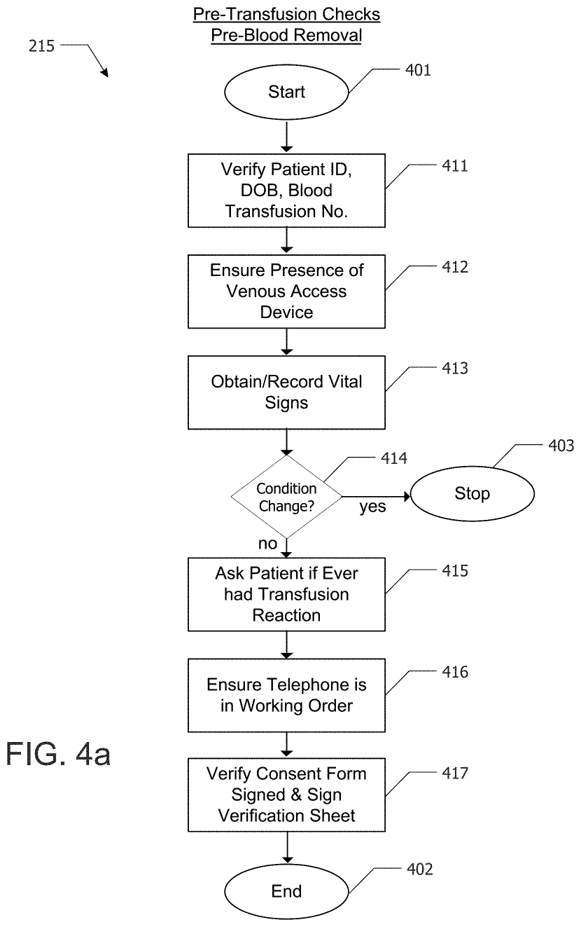

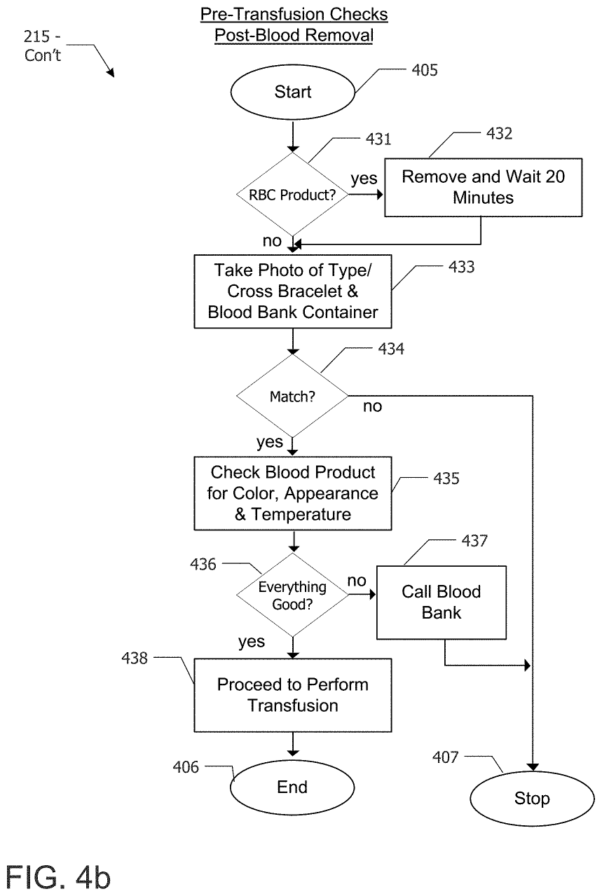

FIGS. 4a-b illustrate flowcharts for an example embodiment of the pre- and post-transfusion checks performed before the blood product removal portion step 215 of a method for providing blood transfusion treatment in a rehabilitation nursing home or skilled care facility according to the present invention. FIG. 4a shows the pre-transfusion checklist performed before the blood product removal portion of a method for providing blood transfusion treatment.

Prior to the removal of blood or blood components from its blood-bank provided storage container, the specially-trained nurse 101 verifies the patient by their full name, DOB, and blood transfusion identification number on the patient's wristband in step 411. In step 412, the nurse 101 ensures the presence of a patient venous access device. Next, the nurse 101 obtains baseline vital signs and records the information on the “out-of-hospital transfusion flowchart sheet” in step 413.

Test step 414 confirms that the patient's condition has not changed from the time the pre-transfusion assessment (I assume this pre-transfusion assessment was done even before the nurse went to the blood bank? When was this mentioned prior to this?) was done, and if it has, the specially-trained nurse 101 stops the procedure 403. When test step 414 confirms that the patient's condition has not changed from the time the pre-transfusion assessment was done, the nurse 101 asks the patient whether or not he/she has ever had a transfusion reaction in step 415. The nurse 101, in step 416, ensures that a telephone is in working order in case of an emergency.

The specially-trained nurse 101 verifies that the “consent for transfusion of blood and blood components” has been signed, and executes a facility nurse verification sheet in step 417 before the process continues to step 215.

FIG. 4b shows the post-transfusion checklist performed before the blood product removal portion of a method for providing blood transfusion treatment. The second portion of step 215 begins when test step 431 determines whether or not red blood cells (RBCs) are to be transfused and if they are, the RBCs are removed in step 431 from the blood bank container's ice approximately twenty (20) minutes prior to infusing to allow slight warming in step 432. Next in step 433, the specially-trained nurse 101 takes a confirmation photo of type and cross bracelet (is this what we've been calling the patient's identification wrist band? Should call it same thing here) along with the local blood bank's marked container to confirm an identification match. Test step 434 determines if there is a match and if not, the procedure stops 407. If test step 434 determines that the information matches, the nurse 101 visually inspects the appearance of the blood component in step 435.

Test step 436 determines if the blood or blood products are abnormal in color or appearance or if the temperature in the storage container has not been maintained. If test step 436 determines any issues, the specially-trained nurse 101 immediately calls the blood bank 140a and the procedure stops 407. If test step 436 determines that the blood or blood component is satisfactory, the process continues to the next step 216.

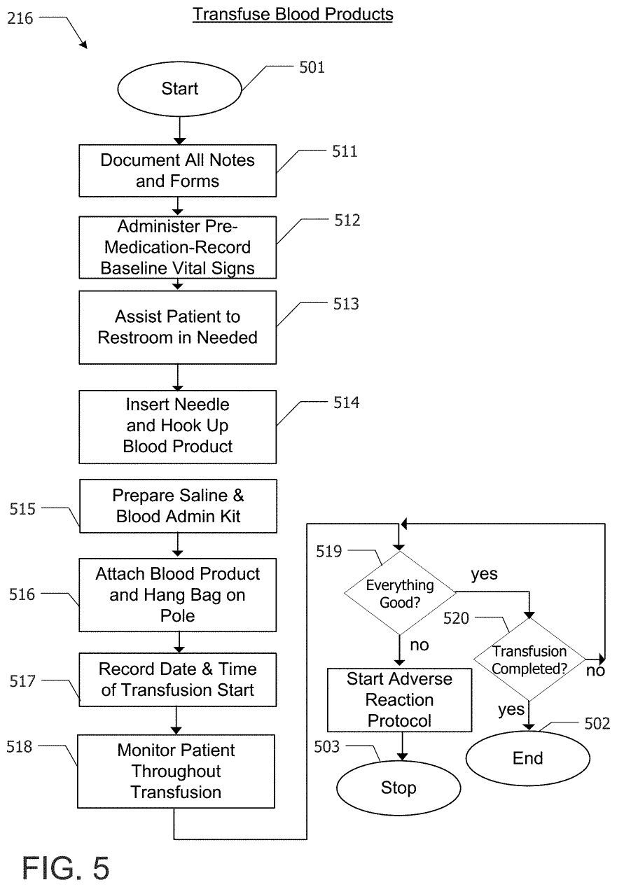

FIG. 5 illustrates a flowchart for an example embodiment of the blood and blood product transfusion portion 216 of a method for providing blood transfusion treatment in a rehabilitation nursing home or skilled care facility according to the present invention. The specially-trained nurse 101 completes the transfusion following protocol written in accordance with health insurance protocol, ImprovePICC (peripherally inserted central catheter) MAGIC, and Centers for Disease Control (CDC) guidelines.

The transfusion begins with step 511, the completion of all notes and forms. The specially-trained nurse's notes shall be completed during the transfusion visit. Transfusion reaction orders shall be reviewed prior to administration of blood components. Blood must not be stored in any container other than that provided by the local blood bank or noted in the notes. A check of the blood component against the “transfusion record” for the unit number, blood type (ABO/Rh), component, and expiration date is performed and documented. The identification number on the patient's wristband is recorded on the: a. “Consent for Transfusion of Blood and Blood Components,” b. “Out-of-Hospital Transfusion Physician's Orders,” c. “Out-of-Hospital Transfusion Assessment,” d. “Out-of-Hospital Transfusion Flow Sheet,” and e. “Transfusion Record.” All of these records are attached to each of the blood components. Finally, all identification attached to the component container should remain attached until the transfusion has been terminated. The specially-trained nurse 101 separates the perforated portion and leaves the rest attached throughout the transfusion.

Next, the specially-trained nurse 101 administers pre-medication, such as a non-aspirin antipyretic, antihistamine and/or a steroid, if ordered, and takes and records a set of baseline vital signs, in step 512. The nurse 10 assists the patient to the restroom or offers the bedpan or urinal in step 513 prior to the start of the transfusion. Infusion of blood components shall be administered intravenously via a central line catheter or peripheral vein. If the peripheral route is used, an 18 to 21 gauge needle is used. A 21 to 23 gauge needle may be used for pediatric patients. A 19 or 20 gauge Huber needle is used for central line catheter access.

A normal saline intravenous (IV) is prepared in step 514 with the blood administration needle set. The needle is inserted as noted above by the specially-trained nurse 101 in step 515. The blood component is attached to the administration tubing using a twisting motion and the blood component is hung onto IV pole in step 516. Throughout the transfusion, the nurse 101 follows the package directions for the use of the blood administration set. The nurse 101 records date and time of the transfusion start in step 517 onto the Facility Verification Sheet for Blood Transfusion.

The specially-trained nurse 101 monitors the patient's condition during the entire transfusion in step 518. If an adverse reaction occurs and is determined in test step 519, the skilled care nurse 101 initiate transfusion reaction procedures, stops the transfusion 503, and contacts the patient's nurse, the facility's director of nursing, the prescribing doctor, and the specially-trained nurse's coordinator or administrator in step 521. If no adverse reaction occurs the transfusion continues until test step 520 determines that all of the blood or blood products have been administered, then the process continues to the next step 217.

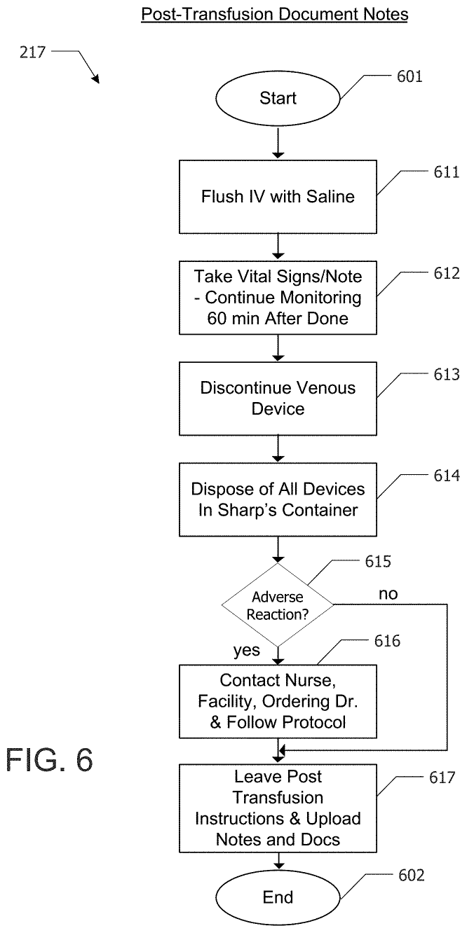

FIG. 6 illustrates a flowchart for an example embodiment of the post-transfusion documentation and notes generation and upload portion step 217 of a method for providing blood transfusion treatment according to the present invention. After all the blood components have been transfused, the specially-trained nurse 101 flushes the IV with normal saline in step 611. Next, the nurse 101 obtains all vital signs at the completion of the transfusion and documents them in step 612. The nurse 101 observes the patient for 60 minutes following the completion of the transfusion to ensure no adverse reactions occur.

During the observation period, the specially-trained nurse 101 discontinues use of the venous access device as indicated in step 613. The e nurse 101 disposes of used access devices, syringes, tubing, etc. in a Sharp's container and returns it to the out-of-hospital transfusion facility for disposal of biohazardous waste in step 614. If an adverse reaction occurs and is determined in test step 615 during the observation period, the skilled nurse 101 initiates transfusion reaction procedures and contacts the patient's nurse, the facility's director or nursing, the prescribing doctor, and the specially-trained nurse's coordinator or administrator in step 616.

The specially-trained nurse 101 leaves post-transfusion instructions with the patient, and/or facility in step 617 and uploads all notes and required documents to the EMR application. These uploaded notes are used by the specially-trained nurse's coordinator or administrator to contact the patient the following day for the post transfusion follow up call. The nurse 101 notifies the facility the transfusion is complete, leaves all completed documents required by the facility, and informs the facility that he/she 101 is leaving as the procedure ends 602.

Even though a particular combination of features is recited in the present application, this combination is not intended to limit the disclosure of the invention. In fact, many of these features may be combined in ways not specifically recited in this application. In other words, any of the features mentioned in this application may be included in this new invention in any combination or combinations to allow the functionality required for the desired operations.

No element, act or instruction used in the present application should be construed as critical or essential to the invention unless explicitly described as such. Further, the phrase “based on” is intended to mean “based, at least in part, on” unless explicitly stated otherwise.

What is claimed:

1. A method for providing remote blood-related healthcare, and more specifically, a method for providing blood transfusion treatment in a rehabilitation nursing home or skilled care facility, the method comprising:
receiving call orders to provide services;
dispatching to a skilled nursing facility to meet a patient;
picking up blood products from a blood bank;
travelling to the skilled nursing facility;
preforming pre-transfusion checks;
transfusing blood products to the patient; and
entering post transfusion document notes.

2. The method according to claim 1, wherein after arriving at the skilled nursing facility, the method further comprising:
checking in with skilled nursing facility;
confirming needed services for the patient;
reviewing the orders maintained at the skilled nursing facility;
greeting the patent and explaining the transfusion procedure;
determining whether any questions regarding required treatment;
requesting additional information regarding questions from skilled nursing facility administrator; and
checking if patent possesses a blood band identification bracelet, and when missing, terminating the transfusion procedure.

3. The method according to claim 1, wherein the preforming pre-transfusion checks comprises:
verifying a patient ID, date-of-birth, and blood transfusion number;
ensuring presence of venous access device;
obtaining and recording vital signs of the patient;
checking patent condition, and when changing, terminating the transfusion procedure;
determining whether patient has previously received a blood transfusion;
ensuring an available telephone is working;
verifying patient consent form signed; and
signing verification documentation.

4. The method according to claim 3, wherein the preforming pre-transfusion checks further comprises:
determining whether red blood cells are being transfused, when red blood cells are being used, remove blood product from transporting contain and ice and wait twenty minutes for the blood product to adjust to room temperature;
taking a photo of patient's type and cross bracelet and blood product container for documentation;
comparing patent type and cross bracelet and blood product container, and, terminating the transfusion procedure when they are in conflict; and
when all pre-checks are completed satisfactorily, performing the transfusion.

5. The method according to claim 4, wherein the performing the transfusion comprises:
documenting all notes and forms;
administering any pre-transfusion medication;
recording baseline patient vital signs;
inserting needle to IV line;
connecting blood product to the IV line;
preparing saline solution and connecting to blood administration kit;
starting transfusion and record date and start time;
monitor patient vital signs until transfusion complete; and
if patient experiences an adverse reaction before the transfusion is complete, terminating the transfusion and initiating adverse reaction protocol.

6. The method according to claim 1, wherein entering post transfusion document notes comprising:
flushing all IV lines with saline;
obtaining and recording vital signs;
discontinuing use of venous device;
disposing all devices used in transfusion in sharps container;
when an adverse reaction has been observed, contacting responsible nurse and ordering doctor, and complete all adverse reaction protocols; and
completing notes and uploading notes and documents to remote server.
